(19) United States
(12) Plant Patent Application Publication
(10) Pub. No.: US 2002/0152516 P1
(43) Pub. Date: Oct. 17, 2002

(54) HYBRID TEA ROSE PLANT NAMED 'POULJOSE'

(75) Inventors: L. Pernille Olesen, Fredensborg (DK); Mogens N. Olesen, Fredensborg (DK)

Correspondence Address: Mogens N. Olesen,, Poulsen Roser Pacific, Inc., 2550 Old Military Road, Central Point, OR, 97502, US

(73) Assignee: Poulsen Roser Aps.

(21) Appl. No.: 09/776,136

(22) Filed: Feb. 1, 2001

(51) Int. Cl. A01H 5/00

(52) U.S. Cl. PLT/133

(57) ABSTRACT

A new Hybrid Tea rose which has abundant, creamy white flowers and attractive foliage. This new and distinct variety has shown to be uniform and stable in the resulting generations from asexual propagation.

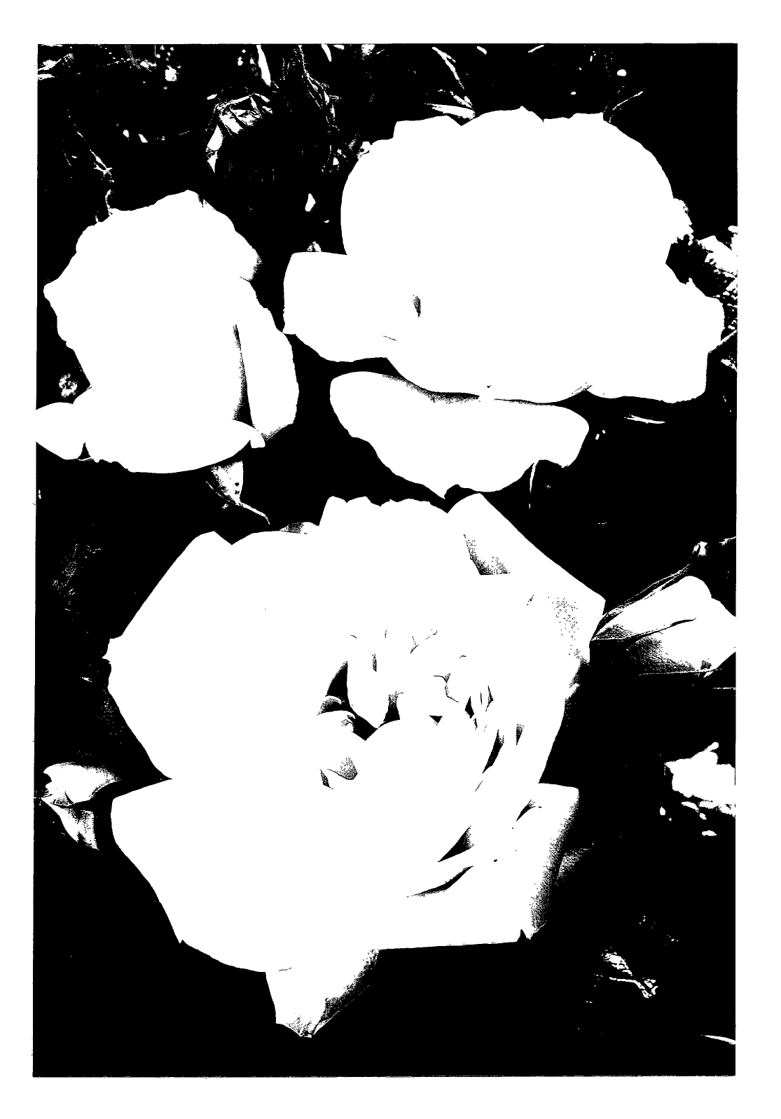

### SUMMARY OF THE INVENTION

The present invention constitutes a new and distinct variety of garden rose plant which originated from a controlled crossing between ‘Polarstern’, an unpatented variety, and ‘POULari’, described and illustrated in U.S. Plant Pat. No. 9,274, issued on Sep. 5, 1994. The two parents were crossed during the summer of 1990 and the resulting seeds were planted in a controlled environment in Fredensborg, Denmark. The new variety is named ‘POULjose’.

The new rose may be distinguished from its seed parent, ‘Polarstern’, by the following combination of characteristics:

1. ‘POULjose’ has fewer petals than ‘Polarstern’.

2. ‘POULjose’ produces a greater quantity of flowers per stem than the seed parent.

3. ‘POULjose’ exhibits more anthocyanin than the seed parent.

The new variety may be distinguished from its pollen parent, ‘POULari’ by the following combination of characteristics:

1. ‘POULjose’ has fewer petals than ‘POULari’.

2. The foliage of ‘POULjose’ is less glossy than the foliage of ‘POULari’.

The objective of the hybridization of this rose variety was to create a new and distinct variety for garden use with unique qualities, such as:

1. Uniform and abundant flowers;

2. Vigorous, but compact growth when propagated both as a budded rose and on its own roots;

3. Disease resistance.

This combination of qualities is not present in previously available commercial cultivars of this type and distinguish ‘POULjose’ from all other varieties of which we are aware.

As part of their rose development program, L. Pernille Olesen and Mogens N. Olesen germinated the seeds from the aforementioned hybridization during the winter of 1990 and conducted evaluations on the resulting seedlings in a controlled environment in Fredensborg, Denmark.

‘POULjose’ was selected in the spring 1991 by the inventors as a single plant from the progeny of the aforementioned hybridization.

Asexual reproduction of ‘POULjose’ by traditional budding and rooted cuttings was first done by L. Pernille and Mogens N. Olesen in their nursery in Fredensborg, Denmark in summer 1991. This initial and other subsequent asexual propagations conducted in controlled environments have demonstrated that the characteristics of ‘POULjose’ are true to type and are transmitted from one generation to the next.

### BRIEF DESCRIPTION OF THE DRAWING

The accompanying color illustration shows as true as is reasonably possible to obtain in color photographs of this type the variety rose ‘POULjose’. Specifically illustrated in SHEET 1 are ‘POULjose's’ foliage, flower buds, partially opened buds, and an open bloom.

### DETAILED DESCRIPTION OF THE VARIETY

The following is a description of ‘POULjose’, as observed in its growth during trials at the testing station Prüfstelle Rethmar under the testing authority Bundessortenamtin in Hannover, Germany and at the breeder's nursery in Fredensborg, Denmark. Color references are made using The Royal Horticultural Society (London, England) Colour Chart, 1995, except where common terms of color are used.

For a comparison, several physical characteristics of the rose variety ‘POULari’, a rose variety from the same inventors described and illustrated in U.S. Plant Pat. No. 9,274 and issued on Sep. 5, 1994 are compared to ‘POULjose’ in Chart 1.

CHART 1

| | ′POULjose′ | ′POULari′ |
|---|---|---|
| Color of open | White Group 155B | White Group 155D |
| flower, outer | | to 157D |
| petals, | | |
| middle zone. | | |
| Petalage | 25-30 | 55-75 |
| Upon opening, | Yellow Group 7D | Greyed-Yellow |
| color basal | | Group 160D |
| petal spot, | | |
| outer side | | |

Parents:

Seed Parent.—‘Polarstern’.

Pollen Parent.—‘POULari’.

Classification:

Botanical.—Rosa hybrida.

Commercial.—Hybrid Tea.

### Flower and Flower Bud

Blooming habit: Recurrent.

Flower bud:

Bud form.—Ovate.

Sepals.—Weak appendages.

Receptacle.—Size: Medium to large.

Peduncle.—Surface: Moderate abundance of hairs and prickles.

Borne.—Singularly.

Flower bloom:

Fragrance.—Weak.

Size.—Medium to large. Average flower diameter is 90 mm when open.

Form.—Shape of flower when viewed from the side: Upper part: Flat. Lower part: Flat. Viewed from above: Irregularly rounded.

Petalage.—Double. Average range: 25-30 petals under normal conditions.

Color:

Petals, Open Bloom.—Inner Side: Middle zone: White Group 155B. Marginal zone: White Group 155B. Basal petal spot: Yellow Group 7D. Outer side: Middle zone: White Group 155B. Marginal zone: White Group 155B. Basal petal spot: Yellow Group 7D.

General tonality: On open flower White Group 155A.

Petals:

Petal Reflex.—Weak.

Undulation of Margin.—Weak.

Size.—Medium.

Reproductive organs:

Filaments.—Color: Yellow.

Hips.—Shape: Pitcher-shaped.

### Plant

Plant growth: Upright to bushy. When grown as a budded field grown plant on Rosa multiflora understock, the average height of the plant is 60-80 cm and the average width is 50 cm.

Stems:

Thorns.—Incidence: Few. Size: Medium. Shape: Concave.

Plant foliage:

Leaf size.—Medium.

Glossy.—Upper side medium glossiness.

Color.—Upper Leaf Surface: Medium green. Juvenile foliage: Exhibits anthocyanin on young shoots. Anthocyanin intonation: Color: Bronze to reddish brown.

Plant leaflets:

Leaflet.—Cross Section: Slight concave. Margin Undulation: Weak to medium. Other: Glossy.

Terminal Leaflet.—Length of blade: Medium. Width of blade: Medium. Shape: Rounded.

Disease resistance: Above average resistance to mildew, rust, black spot, and Botrytis under normal growing conditions in Hannover, Germany and Fredensborg, Denmark.

Cold hardiness: The variety ‘POULjose’ has been found to be cold hardy in Fredensborg, Denmark and Hannover, Germany.

1. A new and distinct variety of rose plant of the hybrid tea rose class, substantially as herein illustrated and described as a distinct and novel rose variety due to its abundant creamy white flowers, disease resistance, and extended period of bloom.
